SEQUENCE LISTING (11 sequences)

SEQ ID NO: 1 - DNA, 900 bp, Bos taurus
  CDS (93)..(899)
ttacaagtcagcagagtctgagcaggtggctcagagcccgctctccagacactgctaccctctctccgagccctgaagtcagaggagccacgatgcctctgccgctggcctgcctgctgcttctcctcctctccacccacagtgccctggccctcaagatctgctccttcaatgtgaggtcctttggggaatccaagaaggcaaactgtaatgccatggatgtcattgtgaaggtcatcaaacgctgtgatatcatactcctgatggaaatcaaggacagcagcaacaggatctgccccacactgatggagaagctaaacggaaattcaagaaaaggcataacatacaactatgtgattagctctcgccttggaagaaacacatataaagaacagtatgcctttctctataaagaaaagctagtgtctgtaaaacaaagctacctctaccacgactatcaggctggagacgcagatgtgttttccagggaaccctttgtggtctggttccagtcaccctacaccgctgtcaaggacttcgtgattgtccccctgcacaccacccctgagacatccgttagagagattgatgagctggctgatgtctacacagatgtgaaacgtcgctggaatgcagagaatttcattttcatgggtgacttcaatgctggctgcagctacgtccccaagaaggcctggaaggacatccgcctgaggacggaccccaagttcgtttggctgatcggggaccaagaggacaccacggtcaagaagagcacaaactgcgcctatgacaggatcgtgcttagaggacaaaatattgtcaactctggtggtcctcaatcaaacctcgtctttgatttccagaaagcttacaggttgtctgaatcgaaggccctgg

SEQ ID NO: 2 - protein, 269 aa, Bos taurus
MPLPLACLLLLLLSTHSALALKICSFNVRSFGESKKANCNAMDVIVKVIKRCDIILLMEIKDSSNRICPTLMEKLNGNSRKGITYNYVISSRLGRNTYKEQYAFLYKEKLVSVKQSYLYHDYQAGDADVFSREPFVVWFQSPYTAVKDFVIVPLHTTPETSVREIDELADVYTDVKRRWNAENFIFMGDFNAGCSYVPKKAWKDIRLRTDPKFVWLIGDQEDTTVKKSTNCAYDRIVLRGQNIVNSGGPQSNLVFDFQKAYRLSESKAL

SEQ ID NO: 3 - DNA, 1212 bp, Homo sapiens
  CDS (145)..(1014)
gaactgaaagttgcaaggagaaatccagtaattcctgttatctgcaggctttataagtcagtggagcctgcggaagtggccagaatccagcactccaagcactgctgtcttctcacagagtcttgaagccagagcagcaccaggatgtcacgggagctggccccactgctgcttctcctcctctccatccacagcgccctggccatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcaaggagaggcataacgtacaactatgtgattagctctcggcttggaagaaacacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtcaagaagagcacaaactgcgcctatgacaggatcgtgcttagaggacaaaatattgtcaactctgttgttcccaagtcaaacagtgtttttgacttccagaaagcttacaagctgactgaagaggaggtaaggttgccatcttgtctttctatgccattgtcctggaaagatgagttagcttgggcaacatgatgagactccatctctatgaaaaattcaaaaaaattagctgggcatggtgatgcatgcctatggtcccagctacttgggaggctgaggtgggaggatcacttgagtccaggaagcggaggttgcagtgagccaagatcgcaccactgcactccaggctgggcaacagagtgagatcctgtctcaaaaaaaaaaaaaaaaaaa

SEQ ID NO: 4 - protein, 288 aa, Homo sapiens
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQNIVNSVVPKSNSVFDFQKAYKLTEEEVRLPSCLSMPLSWKDELAWAT

SEQ ID NO: 5 - protein, 26 aa, Artificial Sequence
  Synthetic Peptide
LKIXSFNVRSFGESKKAGFNAMRVIV

SEQ ID NO: 6 - DNA, 38 bp, Artificial Sequence
  Synthetic DNA
ggccacgcgtcgactagtactttttttttttttttttt

SEQ ID NO: 7 - DNA, 21 bp, Artificial Sequence
  Synthetic DNA
cgtgaggagcttcggcgagag

SEQ ID NO: 8 - DNA, 26 bp, Artificial Sequence
  Synthetic DNA
acaacaggatctgccccatactgatg

SEQ ID NO: 9 - DNA, 25 bp, Artificial Sequence
  Synthetic DNA
tcaactggaaagtggtcgctgacat

SEQ ID NO: 10 - DNA, 27 bp, Artificial Sequence
  Synthetic DNA
acaacaggatctgccccatactgatgg

SEQ ID NO: 11 - DNA, 54 bp, Artificial Sequence
  Synthetic DNA
tggttcttcatgatgatgatgatgatgaactggaaagtggtcgctgacatccag

(19) United States
(12) Patent Application Publication
(10) Pub. No.: US 2005/0049401 A1
(43) Pub. Date: Mar. 3, 2005

(54) Fertility prediction of mammalian males and polyclonal antibodies for the prediction thereof

(75) Inventors: Roy L. Ax, Tucson, AZ (US); Tod C. McCauley, Tucson, AZ (US); George R. Dawson, Tucson, AZ (US); Huanmin Zhang, Okemos, MI (US)

Correspondence Address: OBLON, SPIVAK, MCCLELLAND, MAIER & NEUSTADT, P.C., 1940 DUKE STREET, ALEXANDRIA, VA, 22314, US

(73) Assignee: The Arizona Board of Regents..., Tucson, AZ (US)

(21) Appl. No.: 10/650,791

(22) Filed: Aug. 29, 2003

(51) Int. Cl. C07K 16/18; G01N 33/53; G01N 33/567

(52) U.S. Cl. 530/388.1; 435/7.2

(57) ABSTRACT

The present invention relates to polyclonal antibodies directed to mammalian FAA and methods of detecting the presence of FAA in semen by polyclonal antibody assay. This invention pertains to methods of predicting which candidates within a male mammalian population are likely to be the most fertile, by assaying for the presence and/or localization of FAA by utilizing polyclonal antibodies generated against recombinant FAA.

### BACKGROUND OF THE INVENTION

1. Field of the Invention

The present invention relates to polyclonal antibodies directed to mammalian fertility-associated antigen (FAA) and methods of detecting the presence of FAA in semen by polyclonal antibody assay. This invention pertains to methods of predicting which candidates within a male mammalian population are likely to be the most fertile, by assaying for the presence and/or localization of FAA by utilizing the inventive polyclonal antibodies.

2. Discussion of the Background

Reproductive capacity has a tremendous impact on the animal industry. Reproductive merit is five times more important, economically, than growth performance, and at least ten times more important than product quality, as applied to beef cow/calf producers (Trenkle and Wilham, 1977). Selecting males with the highest fertility potential is one way of improving reproductive efficiency in livestock, and offsetting potentially large economic losses that are incurred as a result of poor fertility. Regrettably, criteria to accurately select males exhibiting the highest fertility are poorly defined, and the physiological importance of parameters that are reliable indicators of fertility potential is poorly understood. Morphological examination of sperm cells and semen, alone, is insufficient to predict a male's reproductive success. Taking even single sire situations and assessing outcomes from artificial insemination, bulls with acceptable semen characteristics by currently accepted evaluations vary widely in their ability to impregnate cows. Variations in fertility between bulls that produce similarly assessed semen contribute greatly to the problem of identifying bulls with the highest fertility potential.

Sperm viability is one of many characteristics commonly evaluated in an attempt to predict fertility. Measurements of motility, acrosomal integrity, cervical mucus penetration and cellular content of DNA, enzymes and lipids are all characteristics that form the general basis for sperm cell viability, and have been studied under different conditions. Unfortunately, correlation of results obtained from this type of analysis and fertility is not reliable. Criteria such as motility, once thought to be highly correlated to fertility, now appear to be less important. This can be explained by a variety of factors, including inaccurate motility characterization, inaccurate measurements of fertility, influence of non-semen factors and perhaps most importantly, effects of other measures on fertility (O'Connor et al., 1981).

Non-return rate is an estimate of relative fertility, calculated by commercial artificial insemination organizations, expressed as a percentage of cows not re-bred to the same bull within a specified time, ordinarily, 30-60 days post-insemination. It is a crude but widely employed reflection of a bull's ability to impregnate cows. The cellular trait most highly correlated with non-return rate is the percentage of intact acrosomes (Saacke and White, 1972) post-thaw.

Another standard test for assessing fertilization capacity, particularly in range beef bulls, is the Breeding Soundness Exam or BSE. This involves determination of scrotal circumference, sperm motility, sperm concentration, sperm morphology and provides a classification of bulls as satisfactory potential breeders if they equal or exceed minimum thresholds (Chenoweth et al., 1992). While BSE is suited for detecting bulls of questionable fertility, it is inadequate to predict differences in fertility among sound breeding bulls that pass the BSE. For example, hundreds of bulls previously accepted as sound breeding sires according to BSE results varied widely in fertility (Bellin et al., 1994, 1996, 1998). The protocols for determining individual elements of the BSE are also tedious, and the BSE is not widely used as a part of bull management techniques when large cross-sections of beef operations are evaluated.

Accordingly, although sperm viability is one requisite trait for determining fertilizing capability, it alone, is not sufficient to identify highly fertile potential breeders. Among sources considered is biochemical assessment of sperm surface components (Graham et al., 1990). An inconsistent relationship between sperm viability characteristics commonly evaluated in semen analysis and fertility has been demonstrated, however, and accordingly, other factors contributing to fertility have been studied. These include comparing the percentage of sperm that undergo an acrosome reaction, the final cellular change in a process called capacitation, in response to treatment with heparin-like glycosaminoglycans (GAG) (Lenz et al., 1988 and Whitfield et al., 1992). These studies have strongly suggested that factors involved in capacitation of mammalian sperm, and in particular, the acrosomal reaction, may be a key to indicating the potential for high fertility.

Capacitation itself is a difficult process to study. Failure to acrosome react, for example, is not clearly indicative of incapacitated sperm, even though sperm are typically considered to have undergone capacitation if their acrosome reacts in response to a specific stimulus. It is generally accepted that capacitation involves substantial modification of sperm surface glycoconjugates (Saling, 1989). Alterations or remodeling of surface components during sperm capacitation are believed to be a pre-requisite to successful fertilization. Competing events involved in capacitation and other pre-fertilization events have led those of skill in the art to identify fertility markers in semen, excluding the results of abnormal semen characteristics. Killian et al. (1993) identified four proteins in bull seminal plasma that were positively correlated with bull fertility. Two-dimensional gel electrophoresis of seminal plasma from bulls differing in fertility by a relatively narrow range indicated that relative density of two proteins (26 kDa, pI 6.2 and 55 kDa, pI 4.5) predominated in highly fertility bulls. Two other proteins were more predominant in seminal plasma from lower fertility bulls. The 55 kDa protein was identified as Osteopontin (Cancel et al., 1997), and the 26 kDa species was identified as Prostaglandin D Synthase (Gerena et al., 1998). Similar analysis of bovine seminal plasma revealed three size classes of heparin and binding proteins (HBPs) of 15-17, 24 and 31 kDa that bound to epididymal sperm in vitro (Miller, et al. 1990). The HBPs in bovine seminal fluid are linked to bull fertility. The 31 kDa HBP was indicated to be correlated with bull fertility potential (Bellin et al., 1994; 1996; 1998). A monoclonal antibody (M1) was generated against high affinity HBP purified from seminal fluid (Bellin et al., 1996).

None of the indicators of potential fertility developed to date are totally satisfactory as a reliable, laboratory method of indicating fertility potential. There are enormous economic gains available by selecting, for breeding, males with potential higher fertility. This is a significant economic issue, as, for every 1,000,000 animals weaned in the United States, a decrease of reproductive efficiency by just one percentage point is equivalent to an economic loss of $4.5 million. In particular, it remains an object of the industry to provide proteins, which, either alone, or considered as a battery, are adequate screening elements to identify males likely to be higher in fertility.

Moreover, the problems associated with fertility and predicting fertility pervade virtually every genus of livestock, including humans. In fact, the impact of infertility and diagnosis thereof in humans takes on a form that cannot be measured in economic loss alone, as for most couples the emotional aspects of infertility can have significant and devastating emotional effects. Accordingly, there exists a critical need to identify new methods and tools to predict enhanced fertility males from large populations of suitable livestock candidates. In addition, there remains a critical need to diagnose, reliably, human male fertility.

Complicating efforts to predict enhanced fertility is the well-documented fact that seminal fluid is a complex mixture consisting of secretions of the male accessory organs of reproduction: seminal vesicles (V.G.), prostate (P.G.), and bulbourethral glands. Of the seminal fluid constituents, some have been shown to inhibit (Davis, 1976; Lenz et al., 1982) and others to stimulate (Florman and First, 1988; Miller et al., 1990; Therien et al. 2001) sperm capacitation in vitro.

Seminal components that stimulate capacitation include a family of heparin-binding proteins (HBPs) that bind to sperm ejaculation and convey heparin-induced capacitation (Miller, 1990). Prior to the present invention, a monoclonal antibody (M1) was available for use in predicting fertility of bulls (see U.S. Patent Application 2001/0008764; the entire contents of which is incorporated herein by reference). However, the murine monoclonal antibody (mAb), M1, generated by immunization with purified HBP, recognized or shared common epitope(s) on three distinct proteins in immunoblots of bovine sperm extracts ranging in size of approximately 20-35 kDa (Bellin et al., 1996, 1998). One of the three HBPs appeared as a single 31-kDa mass and was described as fertility-associated antigen (FAA; McCauley et al., 1999; Bellin et al., 1998). The polynucleotide coding sequence for the HBP designated as FAA, and the amino acid sequence of FAA, is distinctly different from other seminal proteins without or with heparin-binding activity described by others (e.g., Manjunath et al., 1987; Shivaji et al., 1990; Calvette et al., 1996; Therien et al., 2001).

It has been reported (Sprott et al., 2000; Bellin et al., 1994, 1998) that the presence of FAA on bull sperm was associated with 16-19% higher fertility of such bulls used for artificial insemination or natural service than fertility obtained with bulls whose sperm have no detectable FAA. The present inventors found (McCauley et al., 1999) that FAA could be extracted from bull sperm and separated from rest of the HBP complexes by reverse-phase high performance liquid chromatography (RP-HPLC). FAA eluted as a very hydrophobic peptide. Determination of the polynucleotide sequence, preparation of recombinant DNA coding for the recombinant bovine FAA, and evaluation of the biological responses of sperm involved linking numerous steps in an innovative manner and inclusion of other novel steps as set forth in U.S. Patent Application 2002/0048745 (the entire contents of which is incorporated herein by reference).

As stated above, the problems associated with fertility and predicting fertility pervade virtually every genus of livestock, as well as humans. Accordingly, there remains a critical need to identify new methods and tools to predict enhanced fertility males from large populations of suitable candidates.

### SUMMARY OF THE INVENTION

It is an object of the present invention to provide a reliable and reproducible method of predicting fertility of mammalian males.

In one object of the present invention is a polyclonal antibody directed to a mammalian fertility-associated antigen, wherein said fertility-associated antigen is from a mammal selected from the group consisting of buffalo, bovine, human, sheep, goat, rabbit, mouse, horse, pig, dog, cat, camelids, and felids.

In another object of the present invention is a method of producing said polyclonal antibody by injecting a host mammal with the fertility-associated antigen from a mammal other than the host mammal or an antigenic fragment thereof, wherein said fertility-associated antigen may be in a native or a recombinant form, raising a polyclonal antibody specific for said fertility-associated antigen in said host mammal, and recovering said polyclonal antibody from said host mammal. In an embodiment of the present invention, the host mammal is a rabbit, mouse, goat, guinea pig, horse, chicken, donkey, or hamster. Within this object, the mammal may be selected from the group consisting of buffalo, bull, human, rabbit, mouse, horse, sheep, goat, camelids, pig, dog, cat and all felids. To obtain the polyclonal antibody of this object of the present invention, an antisera may be obtained by intravenous extraction or exsanguination. In either case, it may be preferable to purify said polyclonal antibody to substantial purity (≧70%).

In a further object of the present invention is a method of identifying enhanced fertility in a mammalian male out of a group of such males, comprising assaying samples containing semen from each member of said group to determine the presence in said sample of a mammalian fertility-associated antigen, wherein the presence of said protein in a sample is indicative of higher fertility in the donor of said semen sample and wherein said assaying comprises contacting said sample with the polyclonal antibody directed to the fertility-associated antigen or an antisera containing the same. In this object, the amount of binding activity occurring is quantified (i.e., by Western blots, immunofluorescence, ELISA, calorimetric filter binding assay, etc.) and wherein a higher degree of binding is indicative of greater fertility.

In yet another object of the present invention is a sensor for detecting enhanced fertility in a mammalian male comprising a polyclonal antibody (or an antisera containing the same) specific for fertility-associated antigen immobilized on a support matrix. In this object the support matrix is a resin or a membrane, such examples include: nitrocellulose, PVDF, nylon, polystyrene, silica, mixed cellulose ester, or cross-linked agarose. In this respect, the polyclonal antibody may be immobilized by one of several well-appreciated techniques, such as: covalent coupling, electrostatic interaction, hydrogen bonding, or hydrophobic interaction.

In still another object of the present invention is a method of assessing the fertility of a mammalian male by obtaining a sample containing semen from a mammal selected from the group consisting of buffalo, bull, human, sheep, goat, mice, pig, dog, cat, camelids, and felids; processing said sample to obtain seminal plasma proteins or sperm proteins contained therein; contacting said proteins (or appropriately diluted semen sample) to a surface which has been functionally derivatized with a polyclonal antibody specific for a fertility-associated antigen or an antisera containing the same; and detecting an interaction between said proteins and said polyclonal antibody. In this object, the amount of binding activity obtained by said detecting of the sample of a donor is compared to the amount of binding activity occurring for each member of a group of mammalian males to determine the relative presence in semen sample of a mammalian fertility-associated antigen. Further, a higher degree of binding activity for the donor compared to the group of mammalian males is indicative of greater fertility.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1: Western blot of bovine spermatozoal proteins using rabbit antisera produced against recombinant FAA. Lanes 1 and 2 were probed with antisera produced by two different rabbits. The product recognized in each lane represents a 31 kDa sperm protein, identical to the expected molecular mass of FAA.

FIGS. 2A and 2B: Immunolocalization of FAA in bull sperm membranes. Indirect immunofluorescence performed using the FAA-specific rabbit antisera described above demonstrated specific localization of FAA to the acrosome of bull sperm. Sperm were labeled with anti-rFAA antisera and FITC-conjugated goat anti-rabbit secondary antibody. FAA was localized to the acrosomal region with weak labeling of the posterior head.

FIG. 3A-3D: Immunolocalization of FAA in ram sperm membranes. Indirect immunofluorescence performed using the FAA-specific rabbit antisera described above demonstrated specific localization of FAA to the acrosome of ram sperm. Sperm were labeled with anti-rFAA antisera and FITC-conjugated goat anti-rabbit secondary antibody. FAA was localized to the acrosomal region of the head, similar to the pattern of immunofluorescence observed in bull sperm.

FIG. 4A-4D: Immunolocalization of FAA on human sperm. Sperm were labeled with anti-rFAA antisera and FITC-conjugated goat anti-rabbit secondary antibody. Immunofluorescence was variable and predominately apparent in posterior head (PH; panel A, C) and tail principal piece (PP; Panel B). Acrosomal labeling is depicted in panel D. Equatorial segment localization was also detected (data not shown).

FIG. 5: Partial cDNA sequence of bovine FAA spanning a total of 900 bp (SEQ ID NO:1).

FIG. 6: The deduced amino acid (aa) sequence corresponding to the partial cDNA sequence of bovine FAA depicted in FIG. 5. A total of 269 aa are presented (SEQ ID NO:2).

FIG. 7: Partial cDNA sequence of human FAA (SEQ ID NO:3).

FIG. 8: Deduced amino acid sequence of human FAA cDNA sequence given in FIG. 7 (SEQ ID NO:4).

FIG. 9A-9B: Immunolocalization of FAA on porcine sperm. Sperm were labeled with anti-rFAA antisera and FITC-conjugated goat anti-rabbit secondary antibody. Immunofluorescence was variable between males and predominately apparent in the sperm tail and the head.

### DETAILED DESCRIPTION OF THE INVENTION

Unless specifically defined, all technical and scientific terms used herein have the same meaning as commonly understood by a skilled artisan in chemistry, biochemistry, cellular biology, molecular biology and the veterinary and medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

Terms and general procedures used herein:

“Isolated” means separated out of its natural environment.

“Substantial purity” and “substantially pure” means that the level of purity of the substance in question is at least 70%, preferably at least 80% and more preferably at least 90% or at least 95% pure.

“Polynucleotide” in general relates to polyribonucleotides and polydeoxyribonucleotides, it being possible for these to be non-modified RNA or DNA or modified RNA or DNA.

“Polypeptides” are understood as meaning peptides or proteins, which comprise two or more amino acids bonded via peptide bonds. In particular, the term refers to polypeptides with the biological activity of FAA, and also those that are at least 70%, preferably at least 80% and more preferably at least 90% or at least 95% homologous to the polypeptide according to SEQ ID NO:2 or according to SEQ ID NO:4 and have the activity mentioned. Included within the scope of the present invention are polypeptide fragments of SEQ ID NO:2 or of SEQ ID NO:4 or those, which are identical as described herein, which posses the activity of FAA as described herein.

Polynucleotides which encode the FAA as used herein is understood to mean the sequences exemplified in this application as well as those sequences which have substantial identity to SEQ ID NO:1 or to SEQ ID NO:3 and which encode a molecule having one or more of the bioactivities of natural FAA. Preferably, such polynucleotides are those that are at least 70%, preferably at least 80% and more preferably at least 90% or at least 95% homologous to SEQ ID NO:1 or to SEQ ID NO:3.

The terms “stringent conditions” or “stringent hybridization conditions” includes reference to conditions under which a polynucleotide will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing).

Typically, stringent conditions will be those in which the salt concentration is less than approximately 1.5 M Na ion, typically about 0.01 to 1.0 M Na ion (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions also may be achieved with the addition of destabilizing agents such as formamide. Exemplary low stringency conditions include hybridization with a buffer solution of 30 to 35% formamide, 1 M NaCl, 1% SDS (w/v; sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl (Anal. Biochem., 138:267-284, 1984): Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with approximately 90% identity are sought, the Tₘ can be decreased 10° C.

Generally, stringent conditions are selected to be about 5° C. lower than the thermal melting point (Tₘ) for the specific sequence and its complement at a defined ionic strength and pH. However, severely stringent conditions can utilize hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995).

Accomplishment of this invention required polynucleotides that hybridize under stringent conditions to the cDNA encoding bovine FAA. Stringent hybridization conditions are understood to mean those conditions where hybridization, either in solution or on a solid support, occur between two polynucleotide molecules which are 70% to 100% homologous in nucleotide sequence which include 75%, 80%, 85%, 90%, 95%, 98% and all values and subranges therebetween. Methods of stringent hybridization are known in the art which conditions can be calculated by means known in the art. This is disclosed in Sambrook et al., Molecular Cloning: A Laboratory Manual, 2nd Ed., Cold Spring Harbor Laboratory Press, 1989, Cold Spring Harbor, N.Y. and Current Protocols in Molecular Biology, Ausebel et al, eds., John Wiley and Sons, Inc., 2000. Methods of determining percent sequence identity are known in the art, an example of which is the GCG computer sequence analysis software (GCG, Inc, Madison Wis.).

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of DNA sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Suitable vectors for carrying the cDNA of the bovine FAA gene include those vectors which can direct expression of the gene in bacterial, yeast, mammalian and/or insect cells as known in the art. One embodiment of the present invention is whereby the vectors contain an inducible or otherwise regulated expression system whereby the bovine FAA cDNA may be expressed under certain conditions and not expressed under other conditions. Examples of such vectors and suitable cells in which they can be introduced are described in Sambrook et al., Molecular Cloning: A Laboratory Manual, 2.sup.nd Ed., Cold Spring Harbor Laboratory Press, 1989, Cold Spring Harbor, N.Y. and Current Protocols in Molecular Biology, Ausebel et al, eds., John Wiley and Sons, Inc., 2000, the contents of which are herein incorporated by reference. Methods of introducing the cDNA or vector containing the cDNA include calcium mediated transfection, liposomes, electroporation, transformation and infection when the cDNA is contained in a viral vector as known in the art. These and other methods are described in Sambrook et al., Molecular Cloning: A Laboratory Manual, 2nd Ed., Cold Spring Harbor Laboratory Press, 1989, Cold Spring Harbor, N.Y. and Current Protocols in Molecular Biology, Ausebel et al, eds., John Wiley and Sons, Inc., 2000.

Suitable culture conditions for the growth and/or production of the recombinant FAA are dependent on the cell type used. Examples of culture conditions for various cells is described in Sambrook et al., Molecular Cloning: A Laboratory Manual, 2nd Ed., Cold Spring Harbor Laboratory Press, 1989, Cold Spring Harbor, N.Y.; Current Protocols in Molecular Biology, Ausebel et al, eds., John Wiley and Sons, Inc., 2000; and Cells: A Laboratory Manual (Vols. 1-3), Spector et al, eds., Cold Spring Harbor Laboratory Press, Cold Spring Harbor, N.Y., 1988.

Methods of purifying FAA include high performance liquid chromatography (HPLC), ion-exchange chromatography, size exclusion chromatography; affinity separations using materials such as beads with exposed heparin, metals, or lipids; or other approaches known to those skilled in the art. These and other methods of protein purification are disclosed in Sambrook et al., Molecular Cloning: A Laboratory Manual, 2nd Ed., Cold Spring Harbor Laboratory Press, 1989, Cold Spring Harbor, N.Y.; Current Protocols in Molecular Biology, Ausebel et al, eds., John Wiley and Sons, Inc., 2000 and Protein Purification, Scopes and Cantor, eds., Springer-Verlag, 1994 which are incorporated herein by reference.

Since the methods described herein are in vitro diagnostic methods, it is understood that the inventive polyclonal antibody and the inventive antisera containing the polyclonal antibody are extracted from the host source. In this respect, the host source may include: rabbit, mouse, goat, guinea pig, horse, chicken, donkey, or hamster. Further, extraction techniques that may be utilized to obtain the inventive polyclonal antibody and the inventive antisera containing the polyclonal antibody are readily appreciated by the skilled artisan to include: exsanguination and fractionation of egg yolks (e.g., chicken). As such, it is to be understood that the inventive polyclonal antibody and the inventive antisera containing the polyclonal antibody are not “naturally occurring” products of nature.

The fertility-associated antigen (FAA) is strongly correlated with higher fertility bulls. This is reflected in Table 1.

TABLE 1
Relationship of fertility-associated antigen (FAA) on sperm and
pregnancy outcome of bred cows¹

| | |
|---|---|
| Method of detection | Western blot |

| | | |
|---|---|---|
| Fertility-associated antigen (FAA) | Present | Absent |
| Number of Bulls | 304 | 217 |
| Number of cows pregnant/total palpated | 6508/8281 | 3232/5167 |
| Fertility (%) | 78.6 | 62.5 |

¹FAA was detected on sperm by Western blotting of whole sperm extracts. Bulls (2-18 per group) were pastured for 60 d at a ratio of 1 bull:25 cows after segregating on the basis of FAA analysis. Pregnancies were determined by palpation per rectum after (an additional) 60 d post-breeding season.

Purified FAA obtained from a bovine source was obtained according to the methods described in U.S. Patent Application 2001/0008764 (the entire contents of which is incorporated herein by reference), and transferred to a PVDF membrane, and analyzed for N-terminal amino acid sequence. This gave a 26 amino acid sequence LKIXSFNVRSFGESKKAGFNAMRVIV (SEQ ID NO:5) that had a substantial (73%) homology to that of a recently identified deoxyribonuclease I-like protein (DNAS 1 L3) Rodriguez et al., 1997.

Two internal amino acid sequences were generated from lys-C digested FAA. These sequences were 85% and 90% identical to corresponding sequences of the same DNAS protein. FAA is not glycosylated, and is indicated to be a basic peptide with a pI of approximately 8.1. FAA was detected in homogenates of seminal vesicles and bulbourethral glands. FAA extracted from sperm membranes by treatment with hypertonic media was identical to seminal fluid-derived FAA.

Purification of FAA to near homogeneity using heparin affinity and reversed-phase high performance liquid chromatography (RP-HPLC) gave 20 mg HBP per one mL of seminal fluid, 0.5% of which was FAA. A purified fraction eluted from RP-HPLC using a 45% acetonitrile buffer at 26 minutes contained only a single HBP band, as indicated by blotting with M1, at 31 kDa. FAA is indicated to bind to sperm cells and is extractable by treatment with hypertonic media (0.6 N KCl). Analysis of proteins isolated from additional glands, confirmed production in the seminal vesicles and bulbourethral glands, but not in the prostate gland. The mRNA for bovine FAA displayed a consensus sequence between bovine seminal vesicles, prostate, and bulbourethral glands. This suggests that the secreted product does not differ by tissue source. Although the protein was not detected in a limited analysis of prostate extracts, the mRNA was identified in the prostate. FAA has a pI of approximately 8.1. FAA does not contain carbohydrate regions. In the N-terminal amino acid sequence provided, X is an undetermined amino acid, and is likely cysteine.

HBP proteins bind to spermatozoa at ejaculation, and exposure of sperm to seminal fluid HBP mediates capacitation by heparin. HBP complexes with the greatest affinity for heparin contained the 31 kDa FAA protein, as well as the 24 kDa protein discussed below, which are not present in HBP complexes with the least affinity for heparin (Miller et al., 1990). This suggests that the 31 and 24 kDa HBP regulate high affinity heparin binding to sperm. Sperm from high fertility bulls bind heparin with greater affinity than sperm lower fertility bulls (Marks et al., 1985). Therefore, bull semen samples reflecting higher concentration of FAA, alone, or together with TIMP-2 (a 24 kDa heparin-binding protein), indicate that the donors have higher potential fertility, and offer grounds for selection for subsequent breeding.

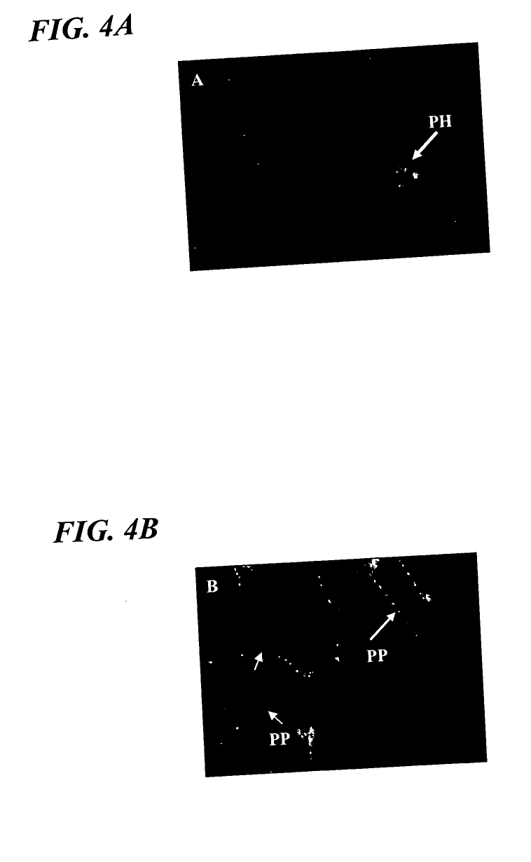

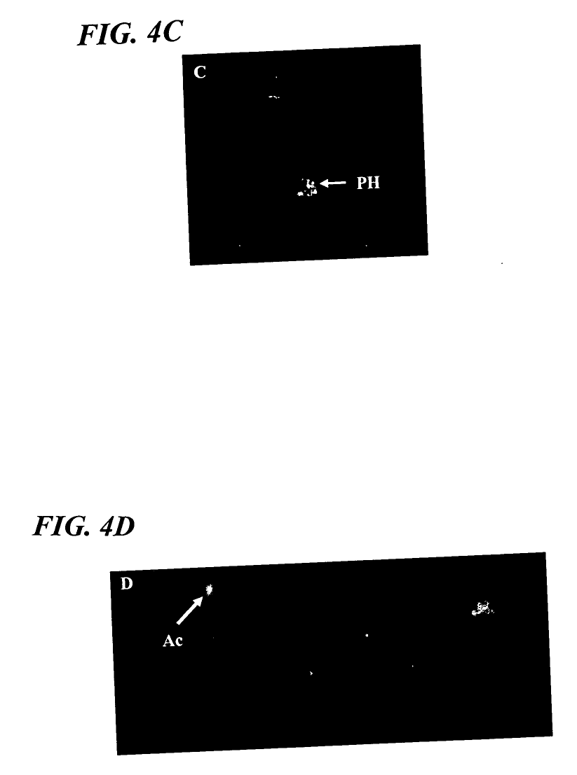

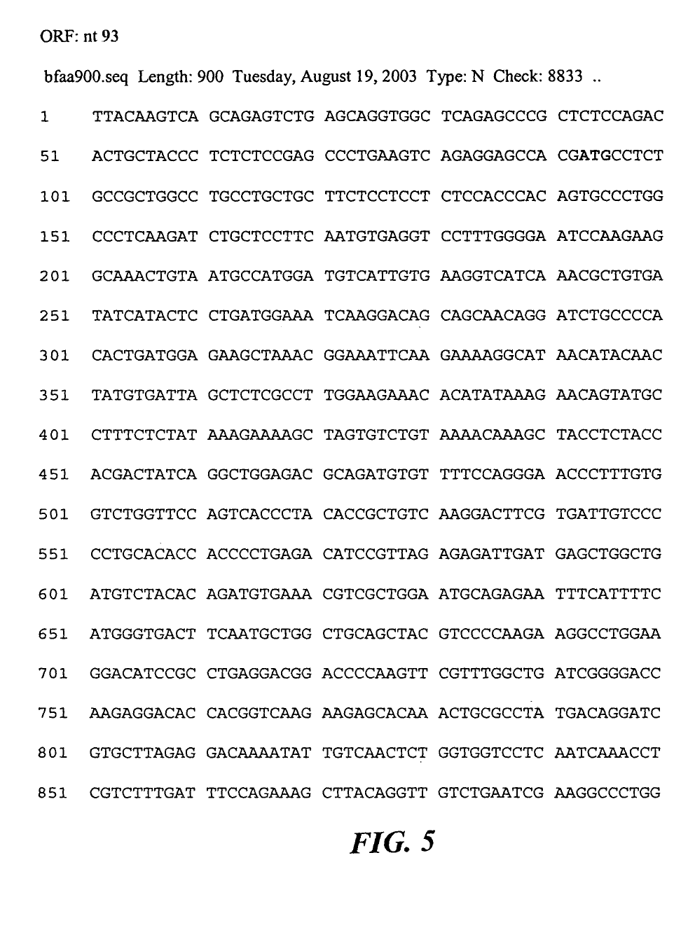

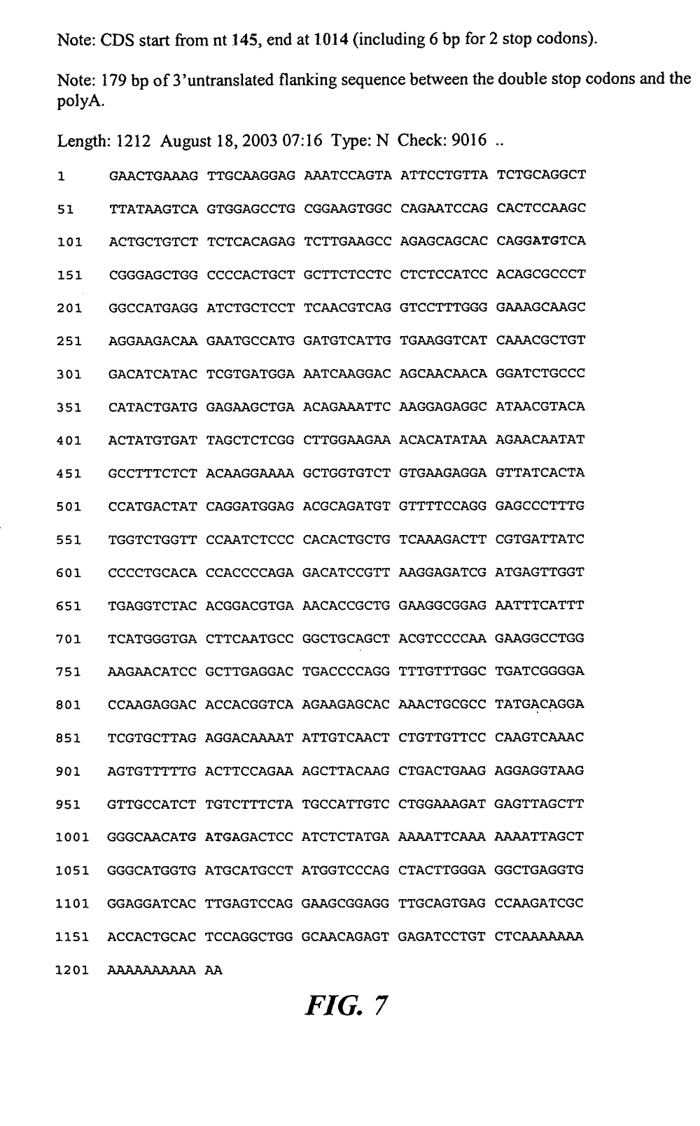

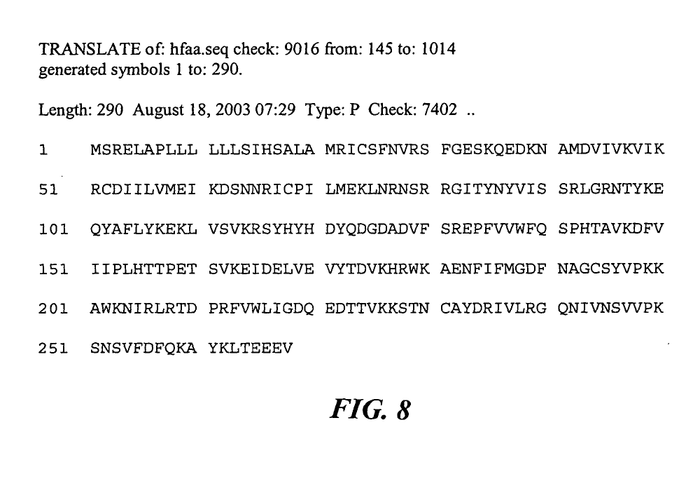

The polynucleotide, and corresponding polypeptide sequences for FAA of several mammals are known. These include: human and bovine. A partial human FAA nucleotide sequence is presented in FIG. 7 and its corresponding deduced amino acid sequence is presented in FIG. 8. As detailed herein and displayed in FIG. 4, FAA has been identified by immunofluorescent analysis in human sperm. The FAA polypeptide has not been detected in horses despite multiple attempts; however, the present inventors have not yet attempted to identify or isolate the FAA gene (polynucleotide) in horses. It is possible that the gene may be present but the protein is not detectable with currently available antibodies. Immunolocalization experiments indicate that an FAA homologue is expressed in ram sperm, gene sequence data have not been acquired in rams to date. Mouse and rat homologues to DNAs IL3 have been identified in the genbank database, suggesting that an FAA homologue is likely to exist in rodents as well.

Prior to the present invention, a monoclonal antibody (M1) was available for use in predicting fertility of bulls. However, the M1 antibody recognized an epitope on multiple proteins in semen, at least 3 peptides ranging from approximately 20-35 kDa. In order to identify the 31 kDa fertility-associated antigen (FAA) using that antibody for diagnostic purposes required SDS-PAGE and Western blotting to separate the admixture of seminal proteins by molecular weight. Accordingly, the pre-existing technology is burdensome, time-consuming, and not as sensitive as desired for widespread industrial applicability.

Hybridoma cell lines, from which the M1 monoclonal antibody was derived, were propagated and tested for production of monoclonal antibodies that recognized only Fertility-Associated Antigen (FAA) in sperm membranes. None of the lines produced sufficient quantities of mono-specific anti-FAA antibody. Therefore, purified native FAA obtained by reverse-phase high performance liquid chromatography (HPLC) was used to immunize a new group of mice. Purified native FAA is extremely hydrophobic and is the last protein in seminal extracts to elute by HPLC. It was not antigenic, presumably due to its overall hydrophobicity.

Therefore, the primary sequence of FAA was analyzed on a software program that reads unique small sequences of amino acids to predict antigenicity. Specifically, the PeptideStructure option in GCG computer sequence analysis software (GCG, Inc, Madison Wis.) was used to evaluate potentially antigenic fragments of bovine FAA. PeptideStructure makes secondary structure predictions for a peptide sequence including measures for antigenicity, hydrophobicity and surface probablility. On a scale of 1 to 5, with 5 most antigenic, 3 fragments had predicted antigenic coefficients exceeding 3.8. Those peptide fragments were custom-synthesized on a contractual basis and were chemically conjugated to bovine serum albumin to produce immunogens. Individual mice were injected with each of those peptide fragments, but they too failed to elicit an immune response. No peptides in semen were detected once antisera collected from the mice were tested in Western blots.

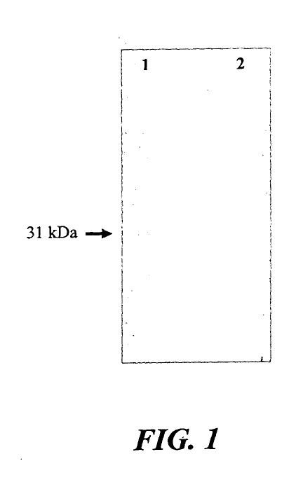

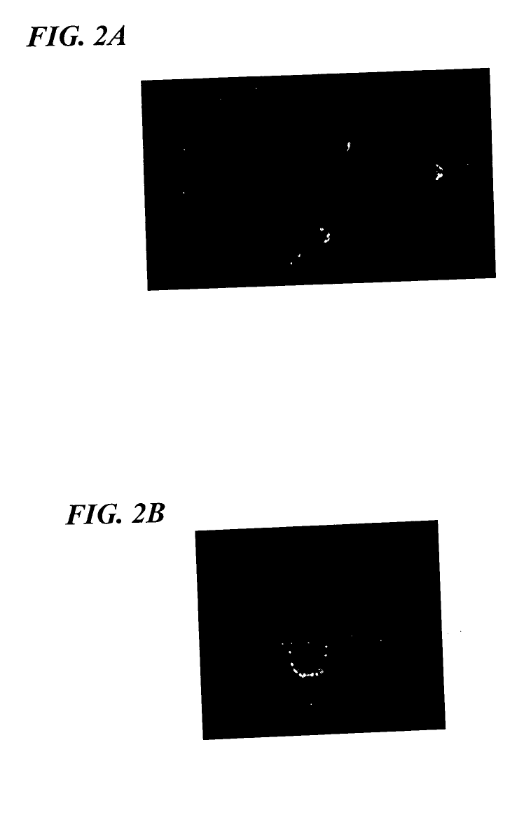

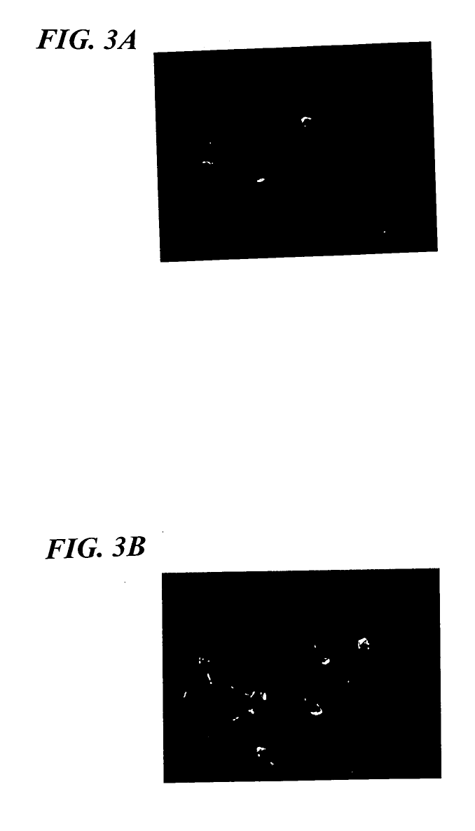

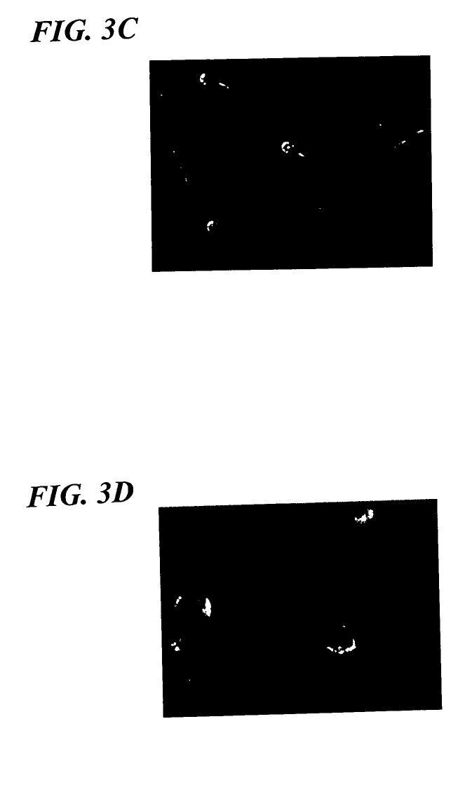

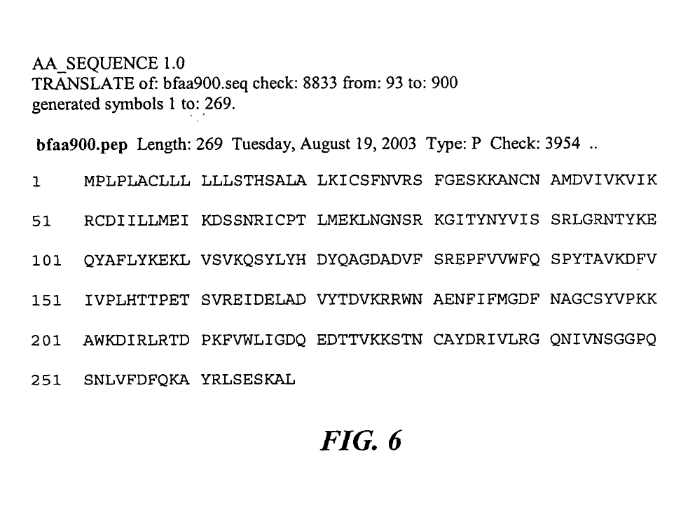

At this point in time, a recombinant FAA had been produced and expressed in E. coli. Native bovine FAA consists of 296 amino acids in its primary sequence (sequence given in FIG. 6; SEQ ID NO:2). The recombinant FAA, spanning residues 73 through 269 of SEQ ID NO:2 (engineered recombinant sequence given in 2002/0048745), had a molecular weight of 22,000 daltons and was designated rFAA-22. rFAA-22, much less hydrophobic than native FAA when purified by HPLC, was used by Strategic BioSolutions (San Diego, Calif.) to immunize rabbits to generate anti-rFAA polyclonal antibodies following the manufacturer's standard protocols. Those antisera were found to be mono-specific for FAA on Western blots of proteins extracted from bovine sperm (see Example 2 and FIG. 1). Immunofluorescence using the antisera demonstrated localization of FAA on the acrosomal cap of bovine sperm (see Example 2 and FIG. 2) and ram sperm (FIG. 3). FAA was localized only to the tail of sperm from one boar and was dispersed in various domains, including the head on sperm from a second boar. Human sperm demonstrated variable localization patterns of FAA including acrosomal, posterior head, tail principal piece and equatorial segment (FIG. 4). With respect to the inventive polyclonal antibody it recognizes an epitope located on the acrosome/acrosomal cap of bull and ram sperm and in the tail and head of boar sperm; that epitope displays variable localization in human sperm that appears to be dependent upon morphological status of the sperm.

As is appreciated in the immunology arts, the antisera of the present invention may be incrementally obtained from the host mammal in small-scale extractions or may be obtained by a final exsanguination of the host mammal. To obtain the polyclonal antibody of this object of the present invention, an antisera may be obtained by intravenous extraction or exsanguination. In either case, it may be preferable to purify said polyclonal antibody to substantial purity (≧70%). Once the antisera has been recovered, it may be processed in any suitable manner including partial purification or addition of stabilizer. The artisan is directed to “Antibodies, A Laboratory Manual”, Cold Spring Harbor, 1988, Harlow and Lane, (ed.) for a description of raising antibodies, including: antigen inoculation, titre monitoring, recovery of antisera, and purification of antisera and/or antibodies contained therein.

Therefore, an embodiment of the present invention is a method of producing a mono-specific anti-FAA polyclonal antibody by injecting a host mammal with an antigenic fragment of the fertility-associated antigen from a mammal other than said host mammal, raising a polyclonal antibody specific for said fertility-associated antigen in said host mammal, and recovering said polyclonal antibody from said host mammal. In this embodiment, the host mammal is preferably a rabbit, a mouse, a goat, a guinea pig, a horse, a chicken, a donkey, or a hamster. Within this embodiment, the mammal may be selected from the group consisting of buffalo, bull, human, rabbit, mouse, horse, sheep, goat, camelids, pig, dog, cat and all felids. As methods of recovering the antisera containing the polyclonal antibodies intravenous extraction and exsanguination may also be mentioned. If desired, within the context of the present invention, the polyclonal antibody may be purified to substantial purity.

In an embodiment of the present invention, the monospecific anti-FAA polyclonal antibodies contained in the antisera raised against the recombinant FAA is purified to substantial purity.

Therefore, in one embodiment of the present invention is a polyclonal antibody, either in a substantially pure form or in a composition, directed to a mammalian fertility-associated antigen, which mammal is a buffalo, a rabbit, a cow, a human, a horse, a sheep, a goat, a mouse, a pig, a dog, a cat, a camelid, and a felid. Preferable, the fertility-associated antigen is from a cow or a human.

In this embodiment, the fertility-associated antigen from bulls has an amino acid sequence as set forth in SEQ ID NO:2 (see FIG. 6). Further in this embodiment the fertility-associated antigen from humans has an amino acid sequence as set forth in SEQ ID NO:4 (see FIG. 8). Within the scope of the present invention are amino acid sequences having at least 70%, preferably at least 80%, more preferably at least 90% or at least 95% homology to the amino acid sequences of SEQ ID NO:2 or SEQ ID NO:4.

As used in the present invention the mono-specific anti-FAA polyclonal antibody may be in a substantially pure form, in an antisera, or may be admixed with suitable buffers, carriers, or other agents so long as the substances admixed thereto do not compromise the mono-specificity. In this context, a substance admixed thereto is considered to compromise the mono-specificity when the binding activity is reduced to 75% or lower when compared to the binding activity of the substantially pure form or the antisera. A substance admixed thereto is also considered to compromise the mono-specificity when the specificity of the anti-FAA polyclonal antibody is reduced to such an extent that it cross-reacts with other proteins other than FAA.

In the present invention, the mono-specific anti-FAA polyclonal antibody, either substantially pure or in the antisera, binds to an epitope on the fertility-associate antigen in a region which is at least 70% homologous to amino acid residues 73-269 of SEQ ID NO:2, preferably at least 80% homologous to amino acid residues 73-269 of SEQ ID NO:2, more preferably at least 90% or at least 95% homologous to amino acid residues 73-269 of SEQ ID NO:2 (see FIG. 6 for native bovine FAA).

In another embodiment of the present invention is a method of identifying enhanced fertility in a mammalian male out of a group of such males, comprising assaying semen samples from each member of said group to determine the presence in said semen sample of a mammalian fertility-associated antigen, wherein the presence of said protein in a semen sample is indicative of higher fertility in the donor of said semen sample and wherein said assaying comprises contacting said semen sample with the mono-specific anti-FAA polyclonal antibody of the present invention, either in substantially pure form, in an antisera, or in a composition. Within this embodiment, it is preferred that the mammalian male is a bull or a human.

Within the context of the present invention, the amount of binding activity occurring is quantified and a higher degree of binding is indicative of greater fertility. As methods of quantification, ELISA, Western blots, immunofluorescence, and colorimetric assay on lateral flow cassette may be mentioned.

In another embodiment of the present invention is a sensor for detecting enhanced fertility in a mammalian male. As used herein, the “sensor” has the polyclonal antibody of the present invention or the antisera containing the polyclonal antibodies of the present invention immobilized on support matrix. It is readily appreciated that any support matrix that is capable of being derivatized for covalent immobilization of the polyclonal antibody of the present invention or the antisera contain the polyclonal antibodies of the present invention can be used. It is further appreciated that any support matrix that is capable of forming a substantial ionic interaction with the polyclonal antibody of the present invention or the antisera containing the polyclonal antibodies of the present invention, such that the ionic interaction is not disrupted by a buffer having an ionic strength of less that 1 M can also be used.

It is preferred that the support matrix of the present invention be a resin or a membrane, including: nitrocellulose, PVDF, nylon, polystyrene, silica, mixed cellulose ester, or cross-linked agarose. It is more preferable that the support matrix be a nitrocellulose membrane or any other membrane filter with an adequate protein binding capacity (i.e., approximately 20 μg/cm²).

In an exemplary embodiment, the support matrix may be a lateral flow cassette as described in U.S. Pat. No. 6,245,577 (the contents of which are incorporated herein by reference). Specifically, the method of the present invention utilizing the lateral flow cassette described in U.S. Pat. No. 6,245,577 would entail, determining the presence of FAA in the biological fluids of mammals. The assay may be performed in a lateral flow cassette or dipstick format where dehydrated, immobilized reagents are spaced along a membrane. A sample of a mammalian biological fluid is exposed first to the labeled anti-FAA antibody. The sample and labeled antibody conjugate move along the membrane via capillary action and are exposed to immobilized anti-FAA antibody at a test position. The presence of FAA in the biological fluid sample is evidenced by a binding of the FAA and labeled antibody to the immobilized antibody resulting in a visualization of the label at the test position (color change). A control reagent of dehydrated antibody may be applied to the membrane at the control position spaced downstream from the test position.

In a particular embodiment of the present invention is a method of assessing the fertility of a mammalian male by using the sensor of the present invention. This method preferably entails:
obtaining a semen sample from a mammal selected from the group consisting of buffalo, bull, human, horse sheep, goat, rabbit, mouse, pig, dog, cat, camelids, and felids,
processing said semen sample to obtain seminal plasma proteins contained therein (including simple dilution of neat semen),
contacting said proteins to a sensor, which has on the surface that is in contact with said proteins the inventive monospecific anti-FAA polyclonal antibodies or an antisera containing the same immobilized thereto (e.g., a surface which has been functionally derivatized with the polyclonal antibody), and
detecting an interaction between said proteins and said polyclonal antibody.

It is particularly preferred that the mammalian male is a bull or a human. Within the context of the present invention, the amount of binding activity occurring is quantified and a higher degree of binding is indicative of greater fertility. As methods of quantification, ELISA, Western blots, colorimetric assay, or immunofluorescence may be mentioned.

In a particularly preferred aspect of this embodiment, the amount of binding activity obtained by said detecting of the semen sample of a donor is compared to the amount of binding activity occurring for each member of a group of mammalian males to determine the relative presence in said semen sample of a mammalian fertility-associated antigen. In this aspect, a higher degree of binding activity for the donor compared to the group of mammalian males is indicative of greater fertility.

Although the present invention is described herein largely with respect to FAA obtained from bull semen, it is understood that the present invention may be broadly applied to other mammals, such as: buffalo, human, horses, sheep, goat, rabbits, mice, pig, dog, cat, carnelids and all felids. With respect to the polyclonal antibody set forth in the examples herein, more specifically the antisera containing polyclonal antibodies directed to the bovine FAA, demonstrates monospecific cross-reactivity with FAA from cows and humans.

It is envisioned that an object of this invention is a method for purifying mammalian FAA from semen, tissue, or cell extracts using anti-FAA affinity chromatography. In this object, the FAA polyclonal antisera is covalently coupled to a solid-phase support matrix (examples of which have been described above) by well-appreciated techniques. Solubilized FAA from semen, tissue, or cell extracts are obtained in either their native state or in a partially purified state. Subsequently, the semen, tissue, or cell extracts are contacted with the immobilized anti-FAA polyclonal antibody to specifically, and reversibly, bind the mammalian FAA present therein to the polyclonal antibody. The FAA:anti-FAA complex is then washed with a low-ionic strength buffer so as to ensure ≧70% (alternatively, ≧80%, ≧90%, or ≧95%) purity. The substantially purified FAA is then recovered from the anti-FAA polyclonal antibody after washing by increasing the ionic strength of the buffer to a concentration adequate to disrupt the FAA:anti-FAA interaction. The suitable ionic strength may be readily determined by the artisan within this object of the invention. Thereby, the present invention provides a new and improved method of obtaining purified native or recombinant FAA from a complex mixture of proteins.

Having generally described this invention, a further understanding can be obtained by reference to certain specific examples, which are provided herein for purposes of illustration only, and are not intended to be limiting unless otherwise specified.

### EXAMPLES

### Example 1

Isolation of Fertility-Associated Antigen (FAA)

Fertility-associated antigen (FAA) was isolated from bovine seminal plasma, sperm membranes, and accessory sex glands. An artificial vagina collected seminal plasma used for protein purification from a vasectomized Holstein bull housed at Sire Power, Inc., Tunkhannock, Pa., frozen in liquid nitrogen, and shipped to the University of Arizona. Seminal plasma was centrifuged at 12,000×g for five min at 4° C. prior to use. Five-hundred μL to 1 mL of seminal fluid supernatant (sperm extracts or accessory sex gland proteins; described below) was injected onto a heparin-affinity column (heparin econo-pac, Bio-Rad, Hercules, Calif.; or an 0.5 cm.times.25 cm column of heparin-sepharose CL-6B, Pharmacia) connected in-line to an ISCO peristaltic pump operating at a flow rate of approximately 1 mL/min. The column was equilibrated with 40 mM Tris-Cl (pH 7.4), 2 mM CaCl₂, 200 μM PMSF, 0.01% NaN₃ (TC-A). Peak detection was achieved by monitoring absorbance at 280 nm with an UA-5 absorbance detector (ISCO, Inc. Lincoln, Nebr.). After achieving baseline conditions, bound protein (HBP) was eluted with 2M NaCl in TC-A, and fractions (15-mL/fraction) were desalted and concentrated by centrifugation (Beckman J-6M, 2,000×g) in centriprep tubes (Ultrafree-15, 12,000 M.Wt. cut-off, Millipore, Bedford, Mass.). All separations took place at 4° C. Unbound and bound fractions were assayed to determine protein concentration (Bio-Rad Dc protein assay, Bio-Rad, Hercules, Calif.), using BSA as standard, then immediately frozen and lyophilized.

Lyophilized powder was resuspended in buffer A (95% H₂O/5% acetonitrile in 0.1% (wt:vol) trifluoroacetic acid [TFA]). Three mg or more of HBP were injected (100 μl-1 mL) onto a C4 reversed-phase HPLC column (Vydac, Hesperia, Calif.) using an Hitachi HPLC autosampler. Proteins were fractionated with a multi-step linear gradient from 25% buffer A to 100% buffer B (70% acetonitrile in 0.085% (wt:vol) TFA) over 55 minutes, with a total run time of 60 min. Thirty-second fractions (1.5 mL/fraction) were collected using a Foxy fraction collector (ISCO, Inc. Lincoln, Nebr.) linked to a diode array detector and dried under vacuum (Speedvac, Savant Instruments, Farmingdale, N.Y.).

Extraction of FAA from Sperm

Frozen-thawed ejaculated sperm were washed 3×in PBS (pH 7.4 with protease inhibitors) to remove seminal plasma, resuspended in 5-mL of PBS/0.6 N KCl, and agitated for 60 min on ice. The sperm suspension was centrifuged at 1,000×g for 10 min, supernatant was removed, and the supernatant containing the KCl extract was recentrifuged at 14,000×g for 30 min. Salt-extracted sperm were washed 3×in PBS, solubilized in an equal volume of 2× sample buffer, and boiled prior to gel electrophoresis. An aliquot of non-extracted ejaculated sperm was solubilized in 2× sample buffer after washing in PBS. The sperm KCl extract was concentrated and desalted by centrifugation, assayed for total protein concentration (Bio-Rad D.sub.c protein assay), and either solubilized in 2× sample buffer or subjected to protein purification as described above.

Accessory Sex Gland Preparation

Seminal vesicles, prostate, and bulbourethral glands were obtained from a bull immediately after slaughter. Tissues were rinsed in cold buffer (50 mM Tris-Cl (pH 7.5) 150 mM NaCl, 2 mM EDTA, 0.5 mM DTT) and homogenized on ice in 50 mM Tris-Cl (pH 7.5), 1% glycerol, 0.2 mM EDTA, 0.5 mM DTT, 0.5 mM PMSF. Total homogenates from each gland were centrifuged at 14,000×g for 60 min and supernatants were subjected to protein purification as described above.

### Example 2

Preparation of Mono-Specific Anti-FAA Antibody

The recombinant form of FAA spanned amino acid residues 73 through 269 of SEQ ID NO:2 encoded by residues 219-807 in the DNA construct (SEQ ID NO:1).

Extraction of Total RNA from Accessory Sex Glands Containing the Messenger RNA for Bovine FAA

Total RNA was extracted from each of the three bull accessory sex glands (seminal vesicles, bulbourethral, and prostate). Fresh tissues of accessory sex glands were harvested from a bull immediately after slaughter. Total RNA was extracted from each gland following a modified protocol (Bonham and Danielpour, 1996). Briefly, one gram of fresh tissue of each gland was placed into 10 ml of solution D (4 M quanidinium thiocyanate, 25 mM sodium acetate, pH 7.0, 5% N-lauroylsarcosine, 0.1 M 2-mercaptoethanol). Each gland was quickly homogenized with a mechanical tissue homogenizer, then chilled on ice while the following buffers were added in a sequential manner: 0.1 vol of 2 M NaAc (pH 4.0), 1.0 vol of water-saturated phenol, 0.2 vol of chloroform-isoamyl alcohol (49:1). After incubating on ice for 15 min, samples were centrifuged at 10,000×g for 20 min at room temperature. The top aqueous phase was then transferred into a new tube containing an equal volume of isopropanol, incubated at −20° C. for 1 h, and centrifuged again at 10,000×g for 20 min at 4° C. to precipitate total RNA. The resulting RNA pellet was then suspended into 0.3 volume of solution D and an equal volume of cold ethanol. This mix was then transferred to a RNeasy column (Qiagen Inc., Chatsworth, Calif.), and centrifuged at 8,000×g for 15 sec. After washing, the final total RNA was eluted with 50 μl of depc-treated water. The total RNA samples were quantified and stored at −70° C.

RT-PCR Amplification: The first strand of cDNA was synthesized with the SuperScript™ II RNase H⁻ RT (GibcoBRL, Grand Island, N.Y.) as catalyst and each of the total RNA samples of bovine seminal vesicle glands, prostate and bulbourethral glands separately as template. Each of the synthesis reactions consisted a mix of 5 μg of total RNA sample with 500 nM adaptor primer (5′-GGC CAC GCG TCG ACT AGT ACT T(16)-3′ (SEQ ID NO:6), Gibco). The reaction mix was heated to 70° C. for 10 min, chilled on ice for 1 min, followed by adding 20 mM Tris-HCl (pH 8.4), 50 mM KCl, 2.5 MgCl₂, 10 mM DTT, and 500 μM dATP, dTTP, dCTP, and dGTP each for a final volume of 20 pI. This mix was incubated at 42° C. for 50 min with 200 units of the SuperScript II RT, and terminated at 70° C. for 15 min. Finally, the mix was incubated with 2 units of RNase H at 37° C. for 20 min to remove the RNA strand of the cDNA:RNA hybrid molecule. These first strand cDNA products were then used as templates in subsequent PCR amplification for isolating the cDNA of the bovine FAA gene. The isolation and identification of the cDNA segments of bovine FAA gene was accomplished by a series of attempts, initiated by a 3′ rapid amplification of cDNA ends (3′RACE) following a commercial protocol (Cat. No. 18373-019, GibcoBRL) with a gene specific primer (CGT GAG GAG CTT CGG CGA GAG (SEQ ID NO:7)) designed based on a N-terminal peptide sequence (LKIXSFNVRSFGESKKAGFNAMRVIV (SEQ ID NO:5)) which was conceived in our laboratory. Based on the 3′ RACE product sequences and a published human cDNA sequence (Rodriguez et al., 1997), which was in high homology to the 3′RACE product sequences, new PCR primers were designed to reamplify the cDNA of the bovine FAA gene. The PCR mix consisted of 50 ng first strand cDNA, 20 mM Tris-HCl (pH 8.3), 50 mM KCl, 1.5 mM MgCl₂, 200 μM DATP, dTTP, dCTP and dGTP each, 400 nM of gene specific primers (5′ primer-ACA ACA ggA TCT gCC CCA TAC TgA Tg (SEQ ID NO:8)) and (3′ primer-TCA ACT GGA AAG TGG TCG CTG ACA T (SEQ ID NO:9), and 0.5 unit of Taq DNA polymerase in a final volume of 20 μl. PCR conditions were 1 min at 94° C., 1 min at 58° C., and 1 min at 72° C. for 35 cycles, followed by a final extension of 72° C. for 30 min.

Cloning of the RT-PCR Product and cDNA Analysis

The fresh PCR products were immediately cloned into the pCR2.10-TOPO vector (Cat. No. k45001-01, Invitrogen) following the manufacturer's instructions. DNA was extracted from the positive clones, air-dried, and resuspended in sterile water at a final concentration of 0.2 μg/μl. Sequence analysis of the RT-PCR products was performed (Applied Biosystems 373 A Automated DNA sequencer utilizing the DyeDeoxy™ terminator chemistry) for each of the clones and for both strands of the inserted DNA. Validation of the cDNA ssequence of the bovine FAA gene was confirmed by analysis of alignments between the deduced peptide sequence of the cloned cDNA and additional two pieces of N-terminal amino acid sequences of internal peptides (a 20-mer and a 15-mer, data not shown), which were generated by lys-C digestion of purified bovine seminal FAA.

Establishment of the Bovine Recombinant Clones

The partial bovine FAA cDNA isolated, identified and validated by 3′RACE, RT-PCR, cloning and sequence analyses, was recloned into the pCR T7/CT-TOPO Expression vector (Cat. No. K421 0-01, Invitrogen, San Diego, Calif.) following the manufacturer's instructions. The vector of the recombinant bovine FAA is a high-level, inducible plasmid vector expressed in E. coli. The insert to establish the recombinant line of clones with the pCR T7/CT-TOPO Expression vector was generated by PCR with redesigned primers (5′ primer: ACA ACA GGA TCT GCC CCA TAC TGA TGG (SEQ ID NO:10)), 3′ primer: TCA TGG TTC TTC ATG ATG ATG ATG ATG ATG AAC TGG AAA GTG GTC GCT GAC ATC CAG (SEQ ID NO:11)) based on the cloned cDNA sequence in pCR2.1-TOPO vectors and our innovation aimed to facilitate the expression of a bio-active recombinant FAA.

Production and Purification of Recombinant Bovine FAA

Transformed One-Shot BL21 (DE3) cells (Invitrogen) were incubated in LB containing 100 μg/ml ampicillin and 34 μg/ml chloramphenicol at 37° C. for 2 h, induced with IPTG at a final concentration of 0.7 mM, continued incubation for an additional 3 h before harvest. The harvested cells were immediately precipitated to pellet by centrifugation at 3,000×g for 15 min at 4° C. and frozen at −20° C. The pellet was then re-suspended in PBS with 0.6 N KCl, and gently agitated for 60 min at 4° C. The pellet suspension was then centrifuged at 600×g for 10 min and the supernatant containing the KCl extract was recovered and centrifuged at 14,000×g for 20 min. The clarified supernatant was applied to a heparin-affinity column (heparin EconoPac, Bio-Rad, Hercules, Calif.; or heparin-Sepharose CL-6B [5×250 mm], Pharmacia) connected in-line to a peristaltic pump (ISCO, Inc. Lincoln, Nebr.) at a flow rate of 1 mL/min. The column was equilibrated with 40 mM Tris (pH 7.4), 2 mM CaCl₂, 200 μM PMSF, 0.01% NaN₃ (TC-A). Peak detection was achieved by monitoring absorbance at 280 nm with an UA-5 absorbance detector (ISCO, Inc. Lincoln, Nebr.). After achieving baseline conditions, recombinant FAA was eluted with 2M NaCl in TC-A. The fraction containing the recombinant FAA was desalted and concentrated by centrifugation (Beckman J-6M, 2,000×g) in centriprep tubes (Ultrafree-15, 12,000 M.Wt. cut-off, Millipore, Bedford, Mass.). All separations took place at 4° C. Samples were assayed to determine protein concentration (Bio-Rad Dc protein assay, Bio-Rad, Hercules, Calif.), using BSA as standard, then immediately frozen and lyophilized until further analysis.

Recombinant FAA from the transformed E. coli cells was further purified using reversed-phase HPLC following a protocol similar to that used to purify native FAA from bull semen. Lyophilized powder was resuspended in buffer A (95% H₂O/5% acetonitrile in 0.1% (wt:vol) trifluoroacetic acid [TFA]). Recombinant FAA was injected (100 μl-1 mL) onto a C4 reversed-phase HPLC column (Vydac, Hesperia, Calif.) using an Hitachi HPLC autosampler. Proteins were fractionated with a multi-step linear gradient from 25% buffer A to 100% buffer B (70% acetonitrile in 0.085% (wt:vol) TFA) over 55 minutes, with a total run time of 60 min. Thirty-second fractions (1.5 mL/fraction) were collected using a Foxy fraction collector (ISCO, Inc. Lincoln, Nebr.) linked to a diode array detector and dried under vacuum (Speedvac, Savant Instruments, Farmingdale, N.Y.).

The inserted DNA included a 592 bp segment of the bovine FAA cDNA corresponding to amino acid residues 72 to 269 of the parent protein. The recombinant bovine FAA contains a total of 215 amino acids. Mass spectral analysis of the rFAA gave a mass of 22 kDa, which differed from the anticipated rFAA mass based on amino acid sequence by less than 3%. The recombinant FAA was much less hydrophobic than native FAA based on different elution times using reversed-phase HPLC. Retention times are correlated with hydrophobicity using reversed-phase HPLC. That lesser hydrophobicity was also confirmed with GCG sequence analysis software using the primary amino acid sequence of the recombinant peptide. The protein expressed had a molecular weight of 22,000 daltons and has been designated rFAA-22.

Production of the Polyclonal Antisera Directed Against Recombinant Bovine FAA

Some of the rFAA-22 was mailed to BioSolutions in San Diego, Calif. and used to immunize two rabbits. Preimmunization bleeds were collected prior to the primary immunization with rFAA-22. The first booster immunization was performed 21 days after the primary immunization. Subsequent booster immunizations were performed at 14 day intervals. Bleeds were performed 9-10 days following booster immunizations. The polyclonal antibody production schedule followed by Strategic Biosolutions is described in the following Table (Table 2).

TABLE 2
STRATEGIC BIOSOLUTIONS - RABBIT 70 DAY PROTOCOL

| | | | | | APPROX. |
|---|---|---|---|---|---|
| DAY | ACTIVITY | DOSE | ROUTE | ADJUVANT | YIELD |
| 0 | PREBLEED | | | | 5 ml |
| 1 | PRIMARY | 200 ug | SQ | CFA | |
| | INJECTION | | | | |
| 21 | BOOST #1 | 200 ug | SQ | IFA | |
| 35 | BOOST #2 | 200 ug | SQ | IFA | |
| 44-45 | BLEED #1 | | | | 15-25 ml |
| 49 | BOOST #3 | 200 ug | SQ | IFA | |
| 58-59 | BLEED #2 | | | | 15-25 ml |
| 63 | BLEED#3 | | | | 15-25 ml |
| 66 | SHIP SERUM | | | | |
| 70 | EXTEND OR | | | | |
| | TERMINATE | | | | |
| | PROJECT | | | | |

Two rabbits responded with a titre and received booster injections. Following those boosters, blood was harvested using standard procedures, and the antisera were found to be mono-specific for FAA on Western blots of proteins extracted from bovine sperm, see FIG. 1.

Biochemical Characterization of the Anti-rFAA Antisera

SDS-PAGE and Western Blotting

Bull semen was collected by electroejaculation, sperm were pelleted by centrifugation (600×g, 15 min at 4° C.) and washed two times in PBS to remove residual seminal plasma proteins. The pellet was resuspended in loading buffer containing Tris (50 mM), P-mercaptoethanol (5%), glycerol (10%), SDS (2%) and PMSF (100 mM) and boiled for 5 min. One-dimensional SDS-PAGE was performed according to the method of Laemmli (1970). Samples were electrophoresed through a 8×10 cm 12% polyacrylamide gel (Mighty Small II, Hoefer Scientific Instruments, San Francisco, Calif.) for approximately 90 min at 20 mA constant current per gel. Proteins were transferred (1 h at 150 mA constant current) using a semi-dry electroblotter (Millipore Milliblot Graphite Electroblotter I) to a 0.2 μm polyvinylidene difluoride (PVDF) membrane (Trans-blot, Bio-Rad) using 10 mM 3-cyclohexylamino-1-propane-sulfonic acid (CAPS) in 10% MeOH as electroblotting buffer. Blotted membranes were blocked with 5% BSA in PBS-T (PBS with 3% Tween) prior to incubation with anti-FAA antisera (1:1000 in PBS-T) for 2 h at room temperature. The FAA antisera was used without purification of immunoglobulins. Preimmune rabbit sera and secondary antibodies alone served as controls. Membranes were washed 3 times in PBS-T and incubated with secondary antibody (mouse anti-rabbit alkaline phosphatase conjugate, Sigma, St. Louis, Mo.) diluted 1:30,000. Blots were developed with an NBT/BCIP colorimetric reaction following manufacturer's instructions. The numbers “I” and “2” on the blot represent the two different rabbits that produced antisera. A comparative analysis of immunolocalization of a fertility related sperm protein (FAA) in bovine, equine, ovine, porcine and human sperm has been performed. FIGS. 2, 3, 4, and 9 are immunolocalization pictures of FAA on bovine, ovine, porcine, and human sperm, respectively.

Indirect Immunofluorescence

Immunolocalization was performed as follows. Ejaculated spermatozoa were washed 3 times in PBS, sperm were permeabilized in cold ethanol for 30 min, mounted on microscope slides and the slides were air-dried. Slides were washed in PBS, nonspecific binding sites were blocked with 10% normal goat serum for 30 min, and anti-FAA antisera was added (1:500) and incubated for 2 h at R.T. After 3 washes in PBS, slides were incubated with FITC-conjugated goat anti-rabbit secondary antibodies (1:1100; Jackson ImmunoResearch Laboratories, West Grove, Pa.) for 45 min. Preimmune rabbit sera and secondary antibodies alone served as controls. Slides were washed in PBS and anti-fade mounting medium was applied. Slides were analyzed with a Leica Diaplan microscope equipped for epifluorescence and images were captured with linked Alphalmager software.

Immunolocalization Results

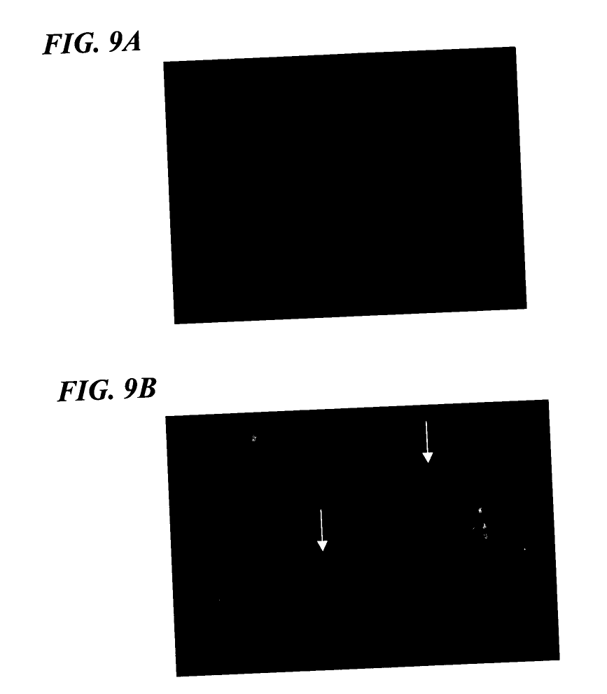

Immunolocalization data illustrated clearly that FAA was localized to the acrosomal domain on bovine (FIG. 2) and ram (FIG. 3) sperm, and boar sperm (FIG. 9). Interestingly, a distinctly different pattern of immunolocalization was observed on human sperm (FIG. 4). FAA localized to the equatorial segment of ejaculated human sperm in one patient and the epitope displayed highly variable localization in human sperm that appeared to be dependent upon or concurrent with the morphology of the sperm cell. Variable acrosomal, posterior head and tail principal piece immunofluorescence have been observed. FAA was not detected on stallion sperm. Fluorescence was undetectable in cauda epididymal human sperm, therefore the FAA epitope is either not expressed or is expressed at undetectable levels compared to ejaculated sperm, consistent with our observation that FAA is produced by the accessory sex glands and binds to sperm at ejaculation. Non-specific labeling with preimmune rabbit sera and secondary antibody controls was minimal, providing evidence that immunofluorescence with post-immune serum was specific. Acrosomal cap localization of FAA in bulls and rams suggests a potential role for FAA in regulating capacitation. Specific tail and head staining in boars suggests a role for FAA in modulating boar sperm function. Distinct localization of FAA to the equatorial segment of human spermatozoa is indicative of a potential regulatory role in sperm-egg interactions. These data are the first to demonstrate specific localization of FAA on bull, ram, boar and human sperm.

### Example 3

Production of a Fertility Detection Sensor

In cooperation with Midland Bioproducts in Boone, Iowa, a lateral-flow cassette (see U.S. Pat. No. 6,245,577; which is incorporated herein by reference) was developed with the antisera sprayed onto the membrane at a 1:1000 dilution. The FAA antisera was preabsorbed to an affinity column containing type-2 tissue inhibitor of metalloproteinases (TIMP-2) covalently attached to a solid support matrix. The TIMP-2 was initially purified from bovine seminal fluid according to the methods of McCauley et al. (2001). The rationale for affinity-purifying the FAA antisera is because TIMP-2 is also a fertility-associated protein present in seminal fluid and is one of three proteins that cross-reacted with the M1 monoclonal antibody (i.e., shared a common epitope with FAA). Therefore, to eliminate any potential cross-reactivity of the FAA polyclonal antisera with TIMP-2, it was felt that affinity purification would be advantageous. Western blots to assay FAA utilizing the polyclonal antisera prior to or following affinity purification on the TIMP-2 column demonstrated a remarkable difference. Those blots probed with preabsorbed polyclonal antisera resulted in bands with better definition and resolution, indicating an enriched specificity compared to polyclonal antisera not preabsorbed.

### Example 4

The fertility detection sensor described in Example 3 was then used to assay FAA status of bulls in 18 herds in Arizona, Nebraska, Iowa and Missouri. Semen was collected by electroejaculation, neat semen was diluted (1:1 up to 1:10 dependent upon sperm density) in buffer (50 mM Tris, 0.3 M NaCl, 5 mM EDTA, 0.1% NaN₃, pH 7.4), and 100 μl of the diluted semen sample was applied to the sample well of a lateral flow cassette containing the FAA antisera sprayed onto the membrane at a 1:1000 dilution. Within approximately 3-5 min the results were determined based on colorimetric development of the test and control bands provided on the cassette. Development of the test band indicated presence of FAA in the semen sample while no development of the test band indicated FAA was not present in the semen sample. Table 3 is a summary of those results.

TABLE 3
FAA Status in Semen from Various Breeds of Bulls in Arizona,
Nebraska, Iowa and Missouri. Results were Obtained Using the
Lateral Flow Cassette with the TIMP-2 pre-absorbed Polyclonal
Antisera.

| | Cassette Field Trial Summary | | | |
|---|---|---|---|---|
| | | No. Bulls | No. Bulls | |
| Herd | No. Bulls | Positive FAA | Negative FAA | Negative (%) |
| 1 | 13 | 13 | 0 | 0 |
| 2 | 33 | 28 | 5 | 15.1 |
| 3 | 114 | 95 | 19 | 16.7 |
| 4 | 51 | 42 | 9 | 17.6 |
| 5 | 5 | 4 | 1 | 20.0 |
| 6 | 19 | 15 | 4 | 21.1 |
| 7 | 85 | 67 | 18 | 21.2 |
| 8 | 58 | 45 | 13 | 22.4 |
| 9 | 75 | 58 | 17 | 22.7 |
| 10 | 40 | 30 | 10 | 25.0 |
| 11 | 80 | 60 | 20 | 25.0 |
| 12 | 33 | 24 | 9 | 27.3 |
| 13 | 19 | 13 | 6 | 31.6 |
| 14 | 138 | 93 | 45 | 32.6 |
| 15 | 53 | 32 | 21 | 39.6 |
| 16 | 15 | 9 | 6 | 40.0 |
| 17 | 59 | 32 | 27 | 45.8 |
| 18 | 24 | 12 | 12 | 50.0 |
| Total | 914 | 672 | 242 | 26.5 ± 2.9 |
| | | | | (SEM) |

As is evident from the data above, 26% of bulls tested overall for FAA in their semen were classified as “negative.”

Table 4 contains a summary of fertility data for 62 bulls used for two consecutive breeding seasons in California. This was a cooperative project with Drs. Cindy and Dave Daley from California State University-Chico (Chico, Calif.). Bulls were pastured at a constant ratio of 25 cows per bull each year. Semen was collected by electroejaculation, neat semen was diluted (1:1) in buffer, and 100 μl of each diluted semen sample were applied to the sample well of a lateral flow cassette as described above. On average, bulls categorized as “positive” for FAA using the lateral flow cassettes produced 35 calves per bull. Herdmates classified as “negative” for FAA only produced 20 calves per bull. Parentage of each calf was verified by DNA fingerprinting.

TABLE 4
Summary of Fertility of Bulls Categorized by Presence or Absence
of FAA Based on Chute-side Cassette Results. Parentage was
Determined by DNA Fingerprinting Analysis. Data were tabulated
for two consecutive breeding seasons.

| | No. | No. | No. | | |
|---|---|---|---|---|---|
| FAA Status | Bulls | Calves | Calves/Bull | No. Cows | Fertility (%) |
| Positive | 50 | 1,734 | 34.7 ± 2.5^{a} | 2,500 | 69.4 |
| Negative | 12 | 244 | 20.3 ± 4.6^{b} | 600 | 40.7 |
| Total | 62 | 1,978 | | 3,100 | 63.8 |

^{a,b}Values with different superscripts differ (P < 0.001, ANOVA).

Summary

Since a mono-specific monoclonal antibody could not be raised, a recombinant fragment of FAA was used as an immunogen to produce mono-specific polyclonal antisera to FAA. The antisera and/or purified polyclonal antibodies were then immobilized on a suitable support matrix and incorporated in a rapid immuno-detection device to enable on-site fertility analysis. In this manner, samples of 914 bulls from 18 herds, 242 animals were determined to lack detectable FAA in their semen. In a field trial with 3,100 cows pasture-bred at a ratio of 25 cows per bull, those bulls categorized as FAA-positive produced 72% more calves compared to their contemporary herdmates classified as FAA-negative.

Numerous modifications and variations on the present invention are possible in light of the above teachings. It is, therefore, to be understood that within the scope of the accompanying claims, the invention may be practiced otherwise than as specifically described herein.

### REFERENCES

Bellin M E, H E Hawkins, and R L Ax. Fertility of range beef bulls grouped according to presence or absence of heparin-binding proteins in sperm membranes and seminal fluid. J Anim Sci 1994; 72:2441-2448.
Bellin M E, Hawkins H E, Oyarzo J N, Vanderboom R J, and R L Ax. Monoclonal antibody detection of heparin-binding proteins on sperm corresponds to increased fertility of bulls. J Anim Sci 1996; 74:173-182.
Bellin M E, Oyarzo J N, Hawkins H E, Zhang H, Smith R G, Forrest D W, Sprott L R, and R L Ax. Fertility-associated antigen on bull sperm indicates fertility potential. J Anim Sci 1998; 76:2032-2039.
Calvette J J, K Mann, L Sanz, M Raida, E Topfer-Petersen. The primary structure of BSP-30K, a major lipid-, gelatin-, and heparin-binding glycoprotein of bovine seminal plasma. FEBS Letter 1996; 399:147-152.
Cancel, A M, Chapman D A, and G J Killian. Osteopontin is the 55-kilodalton fertility-associated protein in holstein bulls seminal plasma. Biol Reprod 1997; 57:1293-1301.
Chenoweth P J, Spitzer J C, and F M Hopkins. A new bull breeding soundness evaluation form. Proc Ann Mtng Soc for Therio, San Antonio, Tex., 1992.
Davis B K. Inhibitory effect of synthetic phospholipid vesicles containing cholesterol on the fertilizing ability of the rabbit. Proc Soc Exp Biol Med 1976; 152:240-243.
Floorman H M and N L First. The Regulation of Acrosomal Exocytosis. II. The zona pellucida-induced acrosome reaction of bovine spermatozoa is controlled by extrinisic positive regulatory elements. Dev Biol 1988; 128:464-473.
Gerena R L, Irakura D, Urade Y, Eguchi N, Chapman D A, and G J Killian. Identification of a fertility-associated protein in bull seminal plasma as lipocalin-type prostaglandin D synthase. Biol Reprod 1998; 58:826-833.
Graham J K, Kunze E, and R H Hammerstedt. Analysis of sperm viability, acrosomal integrity, and mitochondrial function using flow cytometry. Biol Reprod 1990; 43:55-64.
Killian G J, Chapman D A, and L A Rogowski. Fertility-associated proteins in Holstein bull seminal plasma. Bio Reprod 1993; 49:1202-1207.
Lenz R W, R L Ax, H J Grimek, N L First. Proteoglycan from bovine follicular fluid enhances an acrosome reaction in bovine spermatozoa. Biochem Biophys Res Commun. 1982; 106:1092-1098.
Lenz R W, Martin J L, Bellin M E, and R L Ax. Predicting fertility of dairy bulls by inducing acrosome reactions in sperm with chondroitin sulfate. J Dairy Sci 1988; 71:1073-1077.
Manjunath P, M R Siram. Purification and biochemical characterization of three major acidic proteins (BSP-A1, BSP-A2, BSP-A3) from bovine seminal plasma. Biochem J. 1987; 241:685-692.
Marks J L, and R L Ax. Relationship of nonreturn rates of dairy bulls to binding affinity of heparin to sperm. J Dairy Sci 1985; 68:2078-2082.
McCauley T C, H M Zhang, M E Bellin and R L Ax. Purification and characterization of Fertility-Associated Antigen (FAA) in bovine seminal fluid. Mol Reprod Dev 1999; 54:145-153.
McCauley, T. C., H. M. Zhang, M. E. Bellin and R. L. Ax. 2001. Identification of a Heparin-binding Protein in Bovine Seminal Fluid as Tissue Inhibitor of Metalloproteinases-2 (TIMP-2) Mol. Reprod. Dev., 58:336-341.
Miller D J, Winer M A, and R L Ax. Heparin-binding proteins from seminal plasma bind to bovine spermatozoa and modulate capacitation by heparin. Biol Reprod 1990; 42:899-915.
O'Connor M T, Amann R P, and R G Saacke. Comparisons of computer evaluations of spermatozoal motility with standard laboratory tests and their use for predicting fertility. J Anim Sci 1981; 53:1368-1376.
Rodriguez A M, Rodin D, Nomura H, Morton C C, Weremowicz S, and M C Schneider. Identification, localization, and expression of two novel human genes similar to deoxyribonuclease I. Genomics 1997; 42:507-513.
Saacke R G. Semen quality in relation to semen preservation. J Dairy Sci 1983; 66:2635-2644.
Saacke R G and J M White. Semen quality tests and their relationship to fertility. Proc. 4^{th} Tech Conf Artif Insem Reprod., Natl Assoc Anim Breeders. 1972; 2-7.
Saling P M. Mammalian sperm interaction with extracellular matrices of the egg. S R Milligan (Ed.) In: Oxford reviews of reproductive biology. 1989; 339-388. Oxford University Press, New York.
Sprott L R, M D Harris, D W Forrest, J Young, H M Zhang, J N Oyarzo, M E Bellin and R L Ax. Artificial insemination outcomes in beef females using bovine sperm with a detectable fertility-associated antigen. J Anim Sci 2000; 78:795-798.
Thérien I, D Bousquet and P Manjunath. Effect of Seminal Phospholipid-Binding Proteins and Follicular Fluid on Bovine Sperm Capacitation. Biology of Reproduction 2001; 65:41-51.
Trenkle A and R L Wilham Beef Production Efficiency. Science 1977; 198:1009.
Whitfield C H, and T J Parkinson. Relationship between fertility of bovine semen and in vitro induction of acrosome reactions by heparin. Therio 1992; 38:11.
U.S. Patent Application 2001/0008764
U.S. Patent Application 2002/0048745
U.S. Pat. No. 6,245,577

1. A polyclonal antibody directed to a mammalian fertility-associated antigen.

2. The polyclonal antibody of claim 1, wherein said fertility-associated antigen is from a mammal selected from the group consisting of buffalo, rabbit, bovine, human, sheep, goat, mouse, pig, dog, cat, camelids and felids.

3. The polyclonal antibody of claim 1, wherein said fertility-associated antigen is from cow.

4. The polyclonal antibody of claim 3, wherein said fertility-associated antigen has an amino acid sequence as set forth in SEQ ID NO:2.

5. The polyclonal antibody of claim 3, wherein said fertility-associated antigen has an amino acid sequence that is at least 70% homologous to SEQ ID NO:2.

6. The polyclonal antibody of claim 3, wherein said fertility-associated antigen has an amino acid sequence that is at least 80% homologous to SEQ ID NO:2.

7. The polyclonal antibody of claim 3, wherein said fertility-associated antigen has an amino acid sequence that is at least 90% homologous to SEQ ID NO:2.

8. The polyclonal antibody of claim 3, wherein said fertility-associated antigen has an amino acid sequence that is at least 95% homologous to SEQ ID NO:2.

9. The polyclonal antibody of claim 1, wherein said fertility-associated antigen is from human.

10. The polyclonal antibody of claim 9, wherein said fertility-associated antigen has an amino acid sequence as set forth in SEQ ID NO:4.

11. The polyclonal antibody of claim 9, wherein said fertility-associated antigen has an amino acid sequence that is at least 70% homologous to SEQ ID NO:4.

12. The polyclonal antibody of claim 9, wherein said fertility-associated antigen has an amino acid sequence that is at least 80% homologous to SEQ ID NO:4.

13. The polyclonal antibody of claim 9, wherein said fertility-associated antigen has an amino acid sequence that is at least 90% homologous to SEQ ID NO:4.

14. The polyclonal antibody of claim 9, wherein said fertility-associated antigen has an amino acid sequence that is at least 95% homologous to SEQ ID NO:4.

15. The polyclonal antibody of claim 1, wherein said polyclonal antibody is in a substantially pure form.

16. The polyclonal antibody of claim 1, wherein said polyclonal antibody binds to an epitope on the fertility-associated antigen in a region which is at least 70% homologous to amino acid residues 73-269 of SEQ ID NO:2.

17. The polyclonal antibody of claim 1, wherein said polyclonal antibody binds to an epitope on the fertility-associated antigen in a region which is at least 80% homologous to amino acid residues 73-269 of SEQ ID NO:2.

18. The polyclonal antibody of claim 1, wherein said polyclonal antibody binds to an epitope on the fertility-associated antigen in a region which is at least 90% homologous to amino acid residues 73-269 of SEQ ID NO:2.

19. The polyclonal antibody of claim 1, wherein said polyclonal antibody binds to an epitope on the fertility-associated antigen in a region which is at least 95% homologous to amino acid residues 73-269 of SEQ ID NO:2.

20. A method of producing a polyclonal antibody of claim 1 comprising
injecting a host mammal with the fertility-associated antigen from a mammal other than said host mammal or an antigenic fragment thereof,
raising a polyclonal antibody specific for said fertility-associated antigen in said host mammal, and
recovering said polyclonal antibody from said host mammal.

21. The method of claim 20, wherein said host mammal is selected from the group consisting of a mouse, a rabbit, a goat, a guinea pig, a horse, a chicken, a donkey, and a hamster.

22. The method of claim 20, wherein said host mammal is a mouse.

23. The method of claim 20, wherein said host mammal is a rabbit.

24. The method of claim 20, wherein said mammal is selected from the group consisting of buffalo, bovine, human, rabbit, mouse, sheep, goat, pig, dog, cat, camelids, and felids.

25. The method of claim 20, wherein said recovering is by intravenous extraction.

26. The method of claim 20, wherein said recovering is by exsanguination.

27. The method of claim 20, wherein said recovering is by fractionation of egg yolks.

28. The method of claim 20, further comprising purifying said polyclonal antibody to substantial purity.

29. A method of identifying enhanced fertility in a mammalian male out of a group of such males, comprising assaying a sample comprising semen from a source selected from buffalo, bovine, human, rabbit, mouse, sheep, goat, pig, dog, cat, camelids, and felids from each member of said group to determine the presence in said sample of a mammalian fertility-associated antigen, wherein the presence of said protein in said sample is indicative of higher fertility in the donor of said sample and wherein said assaying comprises contacting said sample with the polyclonal antibody of claim 1.

30. The method of claim 29, wherein said mammalian male is a bull.

31. The method of claim 29, wherein said mammalian male is a human.

32. The method of claim 29, wherein the amount of binding activity occurring is quantified, and wherein a higher degree of binding is indicative of greater fertility.

33. The method of claim 32, wherein said quantifying is by at least one method selected from the group consisting of Western blot, immunofluorescence, ELISA and colorimetric binding assay.

34. A sensor for detecting enhanced fertility in a mammalian male comprising the polyclonal antibody of claim 1 immobilized on a support matrix.

35. The sensor of claim 34, wherein said support matrix is a resin or a membrane.

36. A method of assessing the fertility of a mammalian male comprising.
obtaining a sample comprising semen from a mammal selected from the group consisting of buffalo, bull, human, sheep, goat, rabbit, mouse, pig, dog, cat, camelids, and felids,
processing said sample to obtain seminal plasma proteins or sperm proteins contained therein,
contacting said proteins to a surface which has been functionally derivatized with the polyclonal antibody of claim 1, and
detecting an interaction between said proteins and said polyclonal antibody.

37. The method of claim 36, wherein said mammalian male is a bull.

38. The method of claim 36, wherein said mammalian male is a human.

39. The method of claim 36, wherein said detecting is by at least one method selected from the group consisting of Western blot, immunofluorescence, ELISA and calorimetric binding assay.

40. The method of claim 36, wherein the amount of binding activity obtained by said detecting of the sample of a donor is compared to the amount of binding activity occurring for each member of a group of mammalian males to determine the relative presence in said sample of a mammalian fertility-associated antigen.

41. The method of claim 36, wherein a higher degree of binding activity for the donor compared to the group of mammalian males is indicative of greater fertility.

42. An antisera comprising the polyclonal antibody of claim 1.

43. A method of identifying enhanced fertility in a mammalian male out of a group of such males selected from the group consisting of buffalo, bull, human, sheep, goat, rabbit, mouse, pig, dog, cat, camelids, and felids, comprising assaying a sample comprising semen from each member of said group to determine the presence in said sample of a mammalian fertility-associated antigen, wherein the presence of said protein in a sample is indicative of higher fertility in the donor of said sample and wherein said assaying comprises contacting said sample with the antisera of claim 42.

44. The method of claim 43, wherein said mammalian male is a bull.

45. The method of claim 43, wherein said mammalian male is a human.

46. The method of claim 43, wherein the amount of binding activity occurring is quantified, and wherein a higher degree of binding is indicative of greater fertility.

47. The method of claim 46, wherein said quantifying is by at least one method selected from the group consisting of Western blot, immunofluorescence, ELISA and colorimetric binding assay.

48. A method of assessing the fertility of a mammalian male comprising
obtaining a sample comprising semen from a mammal selected from the group consisting of buffalo, bull, human, sheep, goat, rabbit, mouse, pig, dog, cat, camelids, and felids,
processing said sample to obtain seminal plasma proteins or sperm proteins contained therein,
contacting said proteins to a surface which has been functionally derivatized with the antisera of claim 42, and
detecting an interaction between said proteins and said polyclonal antibody.

49. The method of claim 48, wherein said mammalian male is a bull.

50. The method of claim 48, wherein said mammalian male is a human.

51. The method of claim 48, wherein said detecting is by at least one method selected from the group consisting of Western blot, immunofluorescence, ELISA and colorimetric binding assay.

52. The method of claim 48, wherein the amount of binding activity obtained by said detecting of the sample of a donor is compared to the amount of binding activity occurring for each member of a group of mammalian males to determine the relative presence in said sample of a mammalian fertility-associated antigen.

53. The method of claim 48, wherein a higher degree of binding activity for the donor compared to the group of mammalian males is indicative of greater fertility.

54. A method of purifying mammalian FAA from semen, tissue, or cell extracts comprising:
covalently coupling the antisera of claim 42 to a solid-phase support matrix to obtain an immobilized anti-FAA polyclonal antibody;
obtaining a sample containing FAA, wherein said sample is selected from the group consisting of semen, tissue, and cell extracts;
contacting said sample with the immobilized anti-FAA polyclonal antibody to obtain an FAA:anti-FAA complex;
washing the FAA:anti-FAA complex with a low-ionic strength buffer to ensure at least 70% purity of the FAA:anti-FAA complex;
recovering a substantially purified FAA by washing the FAA:anti-FAA complex with a buffer having an ionic strength adequate to disrupt the FAA:anti-FAA interaction.
